(19) United States
(12) Patent Application Publication
(10) Pub. No.: US 2004/0259906 A1
(43) Pub. Date: Dec. 23, 2004

(54) Methods for controlling the proliferation of cells

(76) Inventor: Soner Altiok, Baltimore, MD (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/823,388

(22) Filed: Apr. 12, 2004

Related U.S. Application Data
(60) Provisional application No. 60/461,426, filed on Apr. 10, 2003.

(51) Int. Cl. A61K 31/557

(52) U.S. Cl. 514/299; 514/412; 514/573

(57) ABSTRACT

The invention shows that 12-oxo-phytodienoic acid, coronatine, 6-azido-1-oxo-indanoyl isoleucine and related compounds arrest growth and induce cell death of exponentially proliferating cancer cells and malignant B-cell lymphoma cell lines in a dose dependant manner. It appears that the compounds of the present invention are not toxic to cancer cells but rather irreversibly effect cell growth, differentiation and the interaction between the cancer cell and the extracellular matrix.

### CROSS REFERENCE TO RELATED APPLICATIONS

This application claims priority under 35 U.S.C. § 119(e) to U.S. Provisional Patent Application No. 60/461,426, filed Apr. 10, 2003, the disclosure of which is incorporated herein by reference in its entirety.

### BACKGROUND OF THE INVENTION

1. Field of the Invention

The present invention relates generally to the treatment of disease states that result from uncontrolled cell proliferation, and more specifically to the treatment of cancerous conditions using compounds that activate higher plant defense systems.

2. Discussion of the Related Art

Over the past twenty years only a few drugs isolated from higher plants have yielded clinical agents, the outstanding examples being vinblastine and vincristine from the Madagascan periwinkle, Catharanthus roseus, etoposide, the semi-synthetic lignam, from May-apple Podophyllum peltatum and the diterpenoid taxol, commonly referred to as paclitaxel, from the Pacific yew, Taxus brevifolia. Of these agents, paclitaxel is the most exciting, having received approval by the Food and Drug Administration for the treatment of refractory ovarian cancer. Since the isolation of octadecanoid-derived hormones, there has been a concerted effort by investigators to study other therapeutic applications of octadecanoid-derived hormones their derivatives and compounds that mimic the same.

The oxidative metabolism of polyunsaturated fatty acids in plants gives rise to a group of biologically active compositions known as octadecanoid-derived hormones or oxylipins. Jasmonic acid (“JA”) and 12-oxo-phytodienoic acid (“12-oxo-PDA”) are the two well known active oxylipins in this class, although there is mounting evidence that other octadecanoids are also active. In plants, the oxylipins are involved in a variety of regulatory functions, including growth regulation, wound response, resistance to pathogenic attacks, senescence, fruit ripening, chilling tolerance and tuberization. For example, the oxylipins play a significant and essential role in signal transduction processes between external stimuli (e.g., wounding or pathogenic attack) and the cellular responses thereto (e.g., activation of defense genes such as proteinase inhibitors).

In plants, 12-oxo-PDA (I) is generated by the sequential action of allene oxide synthase and allene oxide cyclase on linoleic acid that has been oxygenated with lipoxygenase.

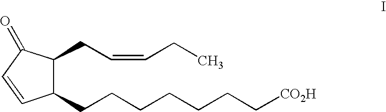

JA (II) can then be synthesized from 12-oxo-PDA by reduction followed by beta-oxidation.

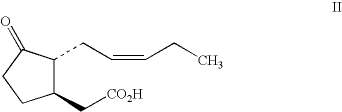

Other compounds similar in structure to 12-oxo-PDA demonstrate phytotoxic effects. Coronatine (III), for example, is a highly active phytotoxin produced by several pathovars of Pseudomonas syrinage (e.g., tomato, glycinea).

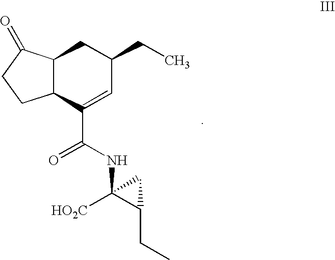

Coronatine, much like 12-oxo-PDA, can activate the stress signaling processes in plants, which, for example, result in volatile emission or tendril coiling. Indeed, coronatine shares both structural and biological properties with 12-oxo-PDA, thus suggesting that coronatine will have the same or similar effects as 12-oxo-PDA if used in a mammalian system.

Similarly, synthetic 6-azido-1-oxo-indanoyl isoleucine (IV) resembles 12-oxo-PDA and coronatine.

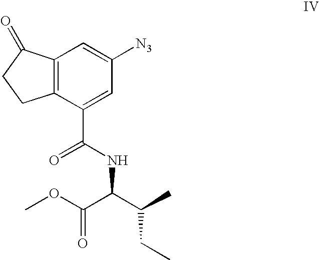

As with coronatine, 6-azido-1-oxo-indanoyl isoleucine (IV) can induce volatile emissions and/or tendril coiling. This compound was synthesized to contain an azido photoaffinity label in order to determine the site of action of coronatine.

The use of JA and its derivatives to affect plant growth and crop improvements has been described. Application of jasmonates can have a wide range of effects on many plant species, ranging from the inhibition of plant development to the promotion of plant processes. For example, it has been shown that use of jasmonic acid ester and giberellin can synergistically enhance plant growth and development. Alternatively, it has been demonstrated that use of particular jasmonates can inhibit sprouting and darkening in tubers after picking. In addition, it has been shown that use of methyl jasmonate helps repel insects.

The levels of JA, 12-oxo-PDA and other intermediates produced during oxylipin synthesis vary considerably among various plant species. These variations in concentration suggest that the relative and absolute concentrations of different oxylipins may provide flexibility within this multifunctional chemical signaling system. Specifically, these findings indicate that there are at least two structurally well-defined processing systems (e.g., receptors and/or binding proteins) that are selective for either of JA or 12-oxo-PDA.

Some protein and signal transduction components involved in cell cycle regulation and apoptosis have been highly conserved among plants and animals during evolution. For example, many plant products (such as taxol) are widely used as growth regulators and apoptotic agents in mammalian systems—especially in the field of cancer treatment. There is still a need, therefore, to identify drug candidates that have activities that are equivalent to or greater than those of known antiproliferative agents.

### SUMMARY OF THE INVENTION

The invention relates generally to the treatment of diseases and disorders that result from uncontrolled cell proliferation. More specifically, this invention provides methods of controlling cell proliferation, comprising administering to a subject in need thereof at least one compound having the Formula V:

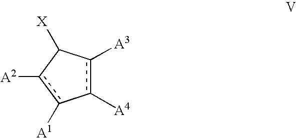

wherein

—is an optional double bond;

A¹ and A² are independently H, Zₘ-OR⁶, oxo, halo, Zₘ-CN, Zₘ-NO₂, azido, Zₘ-NR⁶R⁷, Zₘ-COOR⁶, Zₘ-CONR⁶R⁷, Zₘ-C(═O)R⁶, Zₘ-OC(═O)R⁶, alkyl, allyl, alkenyl, alkynyl, heteroalkyl, heteroallyl, heteroalkenyl, heteroalkynyl, heteroalkoxy, thiol, thioalkyl, Zₘ-cycloalkyl wherein said cycloalkyl is saturated or partially unsaturated, Zₘ-heterocycloalkyl wherein said heterocycloalkyl is saturated or partially unsaturated, or Zₘ-Ar¹, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar¹ may be substituted or unsubstituted;

A³ and A⁴ are independently alkyl, allyl, alkenyl, alkynyl, heteroalkyl, heteroallyl, heteroalkenyl, heteroalkynyl, heteroalkoxy, Zₘ-cycloalkyl wherein said cycloalkyl is saturated or partially unsaturated, Zₘ-heterocycloalkyl wherein said heterocycloalkyl is saturated or partially unsaturated, Zₘ-Ar, Zₘ-O—R⁶, Zₘ-SR⁶, Zₘ-NR⁶R⁷, Zₘ-C(═O)R⁶, Zₘ-OC(═O)R⁶, Zₘ-C(═O)OR⁶, Zₘ-(C═O)NR⁶R⁷, or Zₘ-NHC(═O)R⁶, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted and wherein at least one of A³ or A⁴ is at least three atoms in length;

or A³ and A⁴ together with the atoms to which they are both attached form a substituted or unsubstituted saturated or partially unsaturated ring or a substituted or unsubstituted aromatic ring having at least five atoms, wherein one or more of the atoms is optionally a heteroatom;

R⁶ and R⁷ are independently H, Zₘ-OR⁶, alkyl, allyl, alkenyl, alkynyl, heteroalkyl, heteroallyl, heteroalkenyl, heteroalkynyl, heteroalkoxy, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted;

X is OR⁶, oxo, heteroalkoxy, O-glucosyl, thiol, thioalkyl, NR⁶R⁷, halo, CN, NO₂, or azido;

Ar is aryl or heteroaryl;

Z is CH₂; and

m is an integer between 0 and 10.

In one embodiment, the method comprises administering a compound such as 12-oxo-PDA (I), coronatine (III), 6-azido-1-oxo-indanoyl isoleucine (IV), and related compounds to regulate cell growth and death in proliferating cells such as human malignant neoplastic cells.

The present invention further provides a pharmaceutical composition for controlling proliferative cells in a subject, comprising a therapeutically effective amount of a compound having the formula:

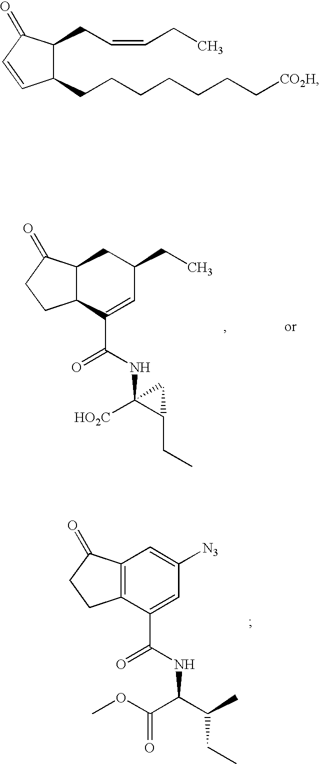

and a pharmaceutically acceptable carrier.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a magnification of breast cancer cells treated with 12-oxo-PDA versus a control sample measured at three and eight-day intervals with arrowed indications of accumulated lipid droplets in the cytoplasm;

FIG. 2 is a magnification of lung cancer cells treated with 12-oxo-PDA versus a control sample measured at three and eight-day intervals with arrowed indications of accumulated lipid droplets in the cytoplasm;

FIG. 3 is a magnification of lung cancer cells treated with various concentrations of 12-oxo-PDA versus a control sample measured after eight days of treatment;

FIG. 4 is a magnification of human malignant diffuse large B-cell lymphoma cells showing morphological changes measured at two and eight-day intervals resulting from treatment with 12-oxo-PDA versus a control sample;

FIG. 5 is a chart illustrating the effect on cell viability for metastatic human breast cancer cells, lung cancer cells and malignant human B-cell lymphoma cells measured after eight days of treatment with 12-oxo-PDA;

FIG. 6 is a chart illustrating the effects on growth and number of metastatic human breast cancer cells, lung cancer cells and malignant human B-cell lymphoma cells in the presence or absence of 12-oxo-PDA;

FIG. 7 is a chart comparing the effects on cell growth and number of human breast cancer cells in the presence of 12-oxo-PDA versus jasmonic acid;

FIG. 8 is a chart comparing the effects on cell growth and number of CRL-2632 lymphoma cells in the presence of 12-oxo-PDA versus jasmonic acid

FIG. 9 is a gel electrophoresis demonstrating that 12-oxo-PDA induces inhibition of cyclin D1 expression and Rb phosphorylation;

FIG. 10 is an amplification of the regulatory effects on cyclin D1 mRNA following treatment with ethanol and 12-oxo-PDA;

FIG. 11 is an amplification of the regulatory effects on p27 mRNA following treatment with ethanol and 12-oxo-PDA;

FIG. 12 is an amplification of the regulatory effects on C-FOS mRNA following treatment with ethanol and 12-oxo-PDA;

FIG. 13 is a gel electrophoresis illustrating the effects of 12-oxo-PDA on apoptosis and inhibits expression of the MCL-1 protein;

FIG. 14 is a graph displaying the growth ratio of CRL-2632 lymphoma cell numbers in the presence or absence of synthetic coronatine (CRNT) over ethanol (control) plates.

FIG. 15 is a gel electrophoresis illustrating the effects of 12-oxo-PDA and coronatine on the expression of the MCL-1 protein; and

FIG. 16 is a gel electrophoresis illustrating the effects of 12-oxo-PDA on beta-tubulin polymerization.

### DETAILED DESCRIPTION OF THE INVENTION

The present invention is directed towards methods of controlling proliferative cells in a subject by administering a therapeutically effective amount of a compound having the Formula V:

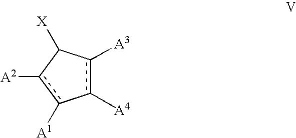

wherein:

—is an optional double bond;

A¹ and A² are independently H, Zₘ-OR⁶, oxo, halo, Zₘ-CN, Zₘ-NO₂, azido, Zₘ-NR⁶R⁷, Zₘ-C(═O)OR⁶, Zₘ-C(═O)NR⁶R⁷, Zₘ-C(═O)R⁶, Zₘ-OC(═O)R⁶, alkyl, allyl, alkenyl, alkynyl, heteroalkyl, heteroallyl, heteroalkenyl, heteroalkynyl, heteroalkoxy, thiol, thioalkyl, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ,-Ar¹ may be substituted or unsubstituted;

A³ and A⁴ are independently alkyl, allyl, alkenyl, alkynyl, heteroalkyl, heteroallyl, heteroalkenyl, heteroalkynyl, heteroalkoxy, Zₘ-cycloalkyl wherein said cycloalkyl is saturated or partially unsaturated, Zₘ-heterocycloalkyl wherein said heterocycloalkyl is saturated or partially unsaturated, Zₘ-Ar, Zₘ-O—R⁶, Zₘ-SR⁶, Zₘ-NR⁶R⁷, Zₘ-C(═O)R⁶, Zₘ-OC(═O)R⁶, Zₘ-C(═O)OR⁶, Zₘ-(C═O)NR⁶R⁷, or Zₘ-NHC(═O)R⁶, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted and wherein at least one of A³ or A⁴ is at least three atoms in length;

or A³ and A⁴ together with the atoms to which they are both attached form a substituted or unsubstituted saturated or partially unsaturated ring or a substituted or unsubstituted aromatic ring having at least five atoms, wherein one or more of the atoms is optionally a heteroatom;

R⁶ and R⁷ are independently H, Zₘ-OR⁶, alkyl, allyl, alkenyl, alkynyl, heteroalkyl, heteroallyl, heteroalkenyl, heteroalkynyl, heteroalkoxy, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted;

X is OR⁶, oxo, heteroalkoxy, O-glucosyl, thiol, thioalkyl, NR⁶R⁷, halo, CN, NO₂, or azido;

Ar is aryl or heteroaryl;

Z is CH₂; and

m is an integer between 0 and 10.

The compounds of this invention can be used to treat or prevent proliferative diseases or disorders in mammals, including but not limited to humans, in which cells grow and increase in number rapidly. For example, the lymphoproliferative disorders are diseases in which there is malignant growth of lymphoid cells and of cells from the reticuloendothelial system (which take up and sequester inert particles). As another example, the myeloproliferative disorders are malignancies of certain bone marrow cells including those that give rise to the red blood cells, the granulocytes (types of white blood cells), and the platelets (crucial to blood clotting).

Proliferative diseases which may be treated or prevented include, but are not limited to, non-small-cell lung cancer, colon, CNS, melanoma, ovarian, renal, prostate and breast cancers, lymphoma, leukemias including acute myelogenous leukemia and chronic myelogenous leukemia, metastatic melanoma, Kaposi's sarcoma, and multiple myeloma.

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl, ethyl, n-propyl, isopropyl, butyl, isobutyl, sec-butyl, tert-butyl, pentyl, isopentyl, tert-pentyl, hexyl, isohexyl, and the like.

The term “alkenyl” refers to linear or branched-chain monovalent hydrocarbon radical of two to twelve carbon atoms, containing at least one double bond, e.g., ethenyl, propenyl, and the like, wherein the alkenyl radical may be optionally substituted independently with one or more substituents described herein, and includes radicals having “cis” and “trans” orientations, or alternatively, “E” and “Z” orientations.

The term “alkynyl” refers to a linear or branched monovalent hydrocarbon radical of two to twelve carbon atoms containing at least one triple bond. Examples include, but are not limited to, ethynyl, propynyl, and the like, wherein the alkynyl radical may be optionally substituted independently with one or more substituents described herein.

The term “allyl” refers to a radical having the formula RC═CHCHR, wherein R is alkyl, alkenyl, alkynyl, cycloalkyl, heterocycloalkyl, aryl, heteroaryl, or any substituent as defined herein, wherein the allyl may be optionally substituted independently with one or more substituents described herein.

The term “cycloalkyl” refers to saturated or partially unsaturated cyclic hydrocarbon radical having from three to twelve carbon atoms, wherein the cycloalkyl may be optionally substituted independently with one or more substituents described herein. The term “cycloalkyl” further includes bicyclic and tricyclic cycloalkyl structures, wherein the bicyclic and tricyclic structures may include a saturated or partially unsaturated cycloalkyl fused to a saturated or partially unsaturated cycloalkyl or heterocycloalkyl ring or an aryl or heteroaryl ring. Examples of cycloalkyl groups include, but are not limited to, cyclopropyl, cyclobutyl, cyclopentyl, cyclohexyl, cycloheptyl, and the like.

The term “heteroalkyl” refers to saturated linear or branched-chain monovalent hydrocarbon radical of one to twelve carbon atoms, wherein at least one of the carbon atoms is replaced with a heteroatom selected from N, O, or S, and wherein the radical may be a carbon radical or heteroatom radical. The heteroalkyl radical may be optionally substituted independently with one or more substituents described herein. The term “heteroalkyl” encompasses alkoxy and heteroalkoxy radicals.

The term “heterocycloalkyl” refers to a saturated or partially unsaturated cyclic radical of 3 to 8 ring atoms in which at least one ring atom is a heteroatom selected from nitrogen, oxygen and sulfur, the remaining ring atoms being C where one or more ring atoms may be optionally substituted independently with one or more substituent described below. The radical may be a carbon radical or heteroatom radical. “Heterocycloalkyl” also includes radicals where heterocycle radicals are fused with aromatic or heteroaromatic rings. Examples of heterocycloalkyl rings include, but are not limited to, pyrrolidine, piperidine, piperazine, tetrahydropyranyl, morpholine, thiomorpholine, homopiperazine, phthalimide, and derivatives thereof.

The term “heteroalkenyl” refers to linear or branched-chain monovalent hydrocarbon radical of two to twelve carbon atoms, containing at least one double bond, e.g., ethenyl, propenyl, and the like, wherein at least one of the carbon atoms is replaced with a heteroatom selected from N, O, or S, and wherein the radical may be a carbon radical or heteroatom radical. The heteroalkenyl radical may be optionally substituted independently with one or more substituents described herein, and includes radicals having “cis” and “trans” orientations, or alternatively, “E” and “Z” orientations.

The term “heteroalkynyl” refers to a linear or branched monovalent hydrocarbon radical of two to twelve carbon atoms containing at least one triple bond. Examples include, but are not limited to, ethynyl, propynyl, and the like, wherein at least one of the carbon atoms is replaced with a heteroatom selected from N, O, or S, and wherein the radical may be a carbon radical or heteroatom radical. The heteroalkynyl radical may be optionally substituted independently with one or more substituents described herein.

The term “heteroallyl” refers to radicals having the formula RC═CHCHR, wherein R is alkyl, alkenyl, alkynyl, cycloalkyl, heterocycloalkyl, aryl, heteroaryl, or any substituent as defined herein, wherein at least one of the carbon atoms is replaced with a heteroatom selected from N, O, or S, and wherein the radical may be a carbon radical or heteroatom radical. The heteroallyl may be optionally substituted independently with one or more substituents described herein.

“Aryl” means a monovalent aromatic hydrocarbon monocyclic radical of 6 to 10 ring atoms or a polycyclic aromatic hydrocarbon, optionally substituted independently with one or more substituents described herein. More specifically the term aryl includes, but is not limited to, phenyl, 1-naphthyl, 2-naphthyl, and derivatives thereof. The term “aryl” further includes heteroaryl rings.

“Heteroaryl” means a monovalent monocyclic aromatic radical of 5 to 10 ring atoms or a polycyclic aromatic radical, containing one or more ring heteroatoms selected from N, O, or S, the remaining ring atoms being C. The aromatic radical is optionally substituted independently with one or more substituents described herein. More specifically the term heteroaryl includes, but is not limited to, furyl, thienyl, pyrrolyl, pyridyl, pyrazolyl, pyrimidinyl, imidazolyl, pyrazinyl, indolyl, thiophen-2-yl, quinolyl, benzopyranyl, thiazolyl, and derivatives thereof.

The term “halo” includes fluoro, chloro, bromo or iodo.

As used herein, the terms “treatment” and “treating” refer to any methodology, regimen, or protocol for affecting or altering a mammalian condition or disease, including, without limitation, (1) preventing a disease or condition from occurring in a subject that may be predisposed to the disease or condition but has not yet been diagnosed with the disease or condition, (2) inhibiting a disease or condition (i.e., arresting the development of the disease or condition), and (3) relieving the disease or condition (i.e., causing a regression of the disease or condition).

In general, the various moieties or functional groups of the compounds of Formulas I and III-IX may be optionally substituted by one or more substituents. Examples of substituents suitable for purposes of this invention include, but are not limited to, halo, alkyl, allyl, alkenyl, alkynyl, heteroalkyl, heteroallyl, heteroalkenyl, heteroalkynyl, heteroalkoxy, Z_{y}-cycloalkyl, Z_{y}-heterocycloalkyl, Z_{y}-OR⁸, Z_{y}-NO₂, Z_{y}-CN, Z_{y}-CO₂R⁸, Z_{y}-(C═O)R⁸, Z_{y}-O(C═O)R⁸, Z_{y}-O-alkyl, Z_{y}-OAr, Z_{y}-SH, Z_{y}-SR⁸, Z_{y}-SOR⁸, Z_{y}-SO₂R⁸, Z_{y}-S—Ar, Z_{y}-SOAr, Z_{y}-SO₂Ar, aryl, heteroaryl, Z_{y}-Ar, Z_{y}-(C═O)NR⁹R¹⁰, Z_{y}-NR⁹R¹⁰, Z_{y}-NR(C═O)R, Z_{y}-SO₂NR⁸R⁹, PO₃H₂, and SO₃H₂, where:

Z is (CH₂),

y is zero or 1,

R⁸, R⁹, and R¹⁰ are independently alkyl, allyl, alkenyl, alkynyl, heteroalkyl, heteroallyl, heteroalkenyl, heteroalkynyl, alkoxy, heteroalkoxy, Z_{y}-cycloalkyl, or Z_{y}-heterocycloalkyl, and

Ar is aryl or heteroaryl,

wherein said alkyl, allyl, alkenyl, alkynyl, heteroalkyl, heteroallyl, heteroalkenyl, heteroalkynyl, alkoxy, heteroalkoxy, Z_{y}-cycloalkyl, Z_{y}-heterocycloalkyl, Ar, R⁸, R⁹, and R¹⁰ may be further substituted or unsubstituted.

In one embodiment, the method comprises administering a compound having Formula V as defined above, wherein A³ and A⁴ are independently

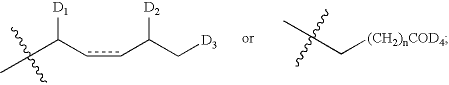

where

n is 3, 4, 5, 6, 7, 8, 9, or 10;

D₁, D₂ and D₃ are independently H, Zₘ-OR⁶, O-glucosyl, heteroalkoxy, thiol, thioalkyl, NR⁷R⁸, halo, CN, NO₂, or azido;

D₄ is H, Zₘ-OR⁶, O-glucosyl, imino, halo, Zₘ-CN, Zₘ-NO₂, azido, Zₘ-C(═O)H, Zₘ-NR⁶R⁷, Zₘ-COOR⁶, Zₘ-CONR⁶R⁷, Zₘ-C(═O)R⁶, Zₘ-OC(═O)R⁶, alkyl, allyl, alkenyl, alkynyl, heteroalkyl, heteroallyl, heteroalkenyl, heteroalkynyl, heteroalkoxy, thiol, thioalkyl, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted,

or D₄ and X, or D₄ and D₃ together form a lactone;

R⁷ and R⁸ are independently H, Zₘ-OR⁶, alkyl, allyl, alkenyl, alkynyl, heteroalkyl, heteroallyl, heteroalkenyl, heteroalkynyl, heteroalkoxy, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted; and

Zₘ, Ar, R⁶ and R⁷ are defined as above.

In another embodiment, the method comprises administering a compound having Formula V as defined above, wherein A³ and A⁴ are independently

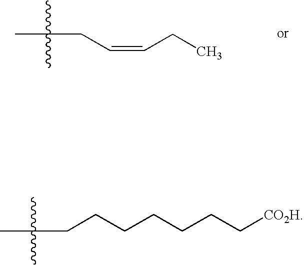

Another aspect of this invention provides a method of controlling proliferative cells in a subject, comprising administering a compound having the Formula VI:

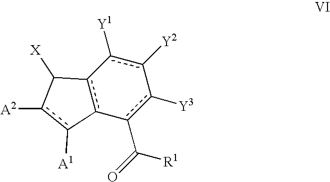

Wherein R¹ is

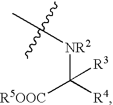

R², R³, R⁴ and R⁵ are independently H, Zₘ-OR⁶, alkyl, allyl, alkenyl, alkynyl, heteroalkyl, heteroallyl, heteroalkenyl, heteroalkynyl, heteroalkoxy, Zₘ-NR⁶R⁷, Zₘ-COOR⁶, Zₘ-CONR⁶R⁷, Zₘ-C(═O)R⁶, Zₘ-OC(═O)R⁶, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted,

or R³ and R⁴ together with the atoms to which they are both attached form a saturated or partially unsaturated ring, wherein said saturated ring or partially unsaturated ring may be substituted or unsubstituted; and

Y¹, Y², and Y³ are independently H, Zₘ-OR⁶, alkyl, allyl, alkenyl, alkynyl, heteroalkyl, heteroallyl, heteroalkenyl, heteroalkynyl, heteroalkoxy, ZₘNR⁶R⁷, Zₘ-COOR⁶, Zₘ-CONR⁶R⁷, Zₘ-C(═O)R⁶, Zₘ-OC(═O)R⁶, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₙ-heterocycloalkyl, and Zₘ-Ar may be substituted or unsubstituted;

and wherein A¹, A², X, Zₘ, Ar, R⁶ and R⁷ defined as above.

In one embodiment, R¹ is a substituted or unsubstituted natural amino acid, including, but not limited to alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine; or an unnatural amino acid, including, but not limited to 4-hydroxyproline, hydroxylysine, demosine, isodemosine, 3-methylhistidine, norvaline, beta-alanine, gamma-aminobutyric acid, cirtulline, homocysteine, homoserine, omithine and methionine sulfone.

In one embodiment, the method uses a compound having the Formula VII or VIII:

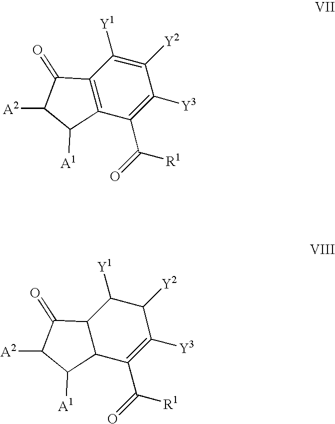

where A¹, A², Y¹, Y², Y³, and R¹ are defined as above. In one specific embodiment, the method uses a compound having the Formula IX:

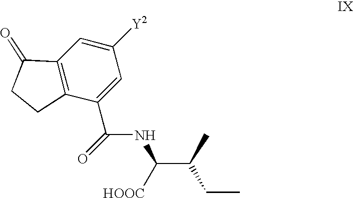

where Y² is defined as above. In a specific embodiment, Y² is azido.

Another aspect of this invention provides a method of controlling proliferative cells in a subject, comprising administering a compound having the Formula I

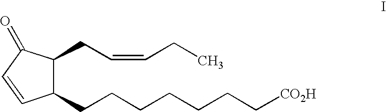

Yet another aspect of this invention provides a method of controlling proliferative cells in a subject, comprising administering a compound having the Formula III

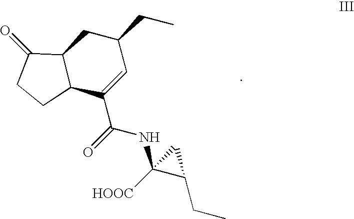

The compounds of this invention may possess one or more asymmetric centers; such compounds can therefore be produced as individual (R)— or (S)— stereoisomers or as mixtures thereof. Unless indicated otherwise, the description or naming of a particular compound in the specification and claims is intended to include both individual enantiomers and mixtures, racemic or otherwise, thereof. Accordingly, this invention also includes racemates and resolved enantiomers, and diastereomers compounds of the Formulas I and III-IX. The methods for the determination of stereochemistry and the separation of stereoisomers are well known in the art (see discussion in Chapter 4 of “Advanced Organic Chemistry”, 4th edition J. March, John Wiley and Sons, New York, 1992).

In order to use a compound of the Formula I or III-IX, or a pharmaceutically acceptable salt or in vivo cleavable prodrug thereof, for the therapeutic treatment (including prophylactic treatment) of mammals including humans, it is normally formulated in accordance with standard pharmaceutical practice as a pharmaceutical composition. According to this aspect of the invention there is provided a pharmaceutical composition that comprises a compound of the Formula I or III-IX, or a pharmaceutically acceptable salt or in vivo cleavable prodrug thereof, as defined hereinbefore in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of a compound of this invention that is combined with one or more excipients to produce a single dosage form will necessarily vary depending upon the host treated and the particular route of administration. For example, a formulation intended for oral administration to humans will may contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

The size of the dose for therapeutic or prophylactic purposes of a compound of Formula I or III-IX will naturally vary according to the nature and severity of the conditions, the age and sex of the animal or patient and the route of administration, according to well known principles of medicine. For example, the method may comprise at least one of an hourly administration, a daily administration, a weekly administration, or a monthly administration of one or more compositions described herein.

In addition to compounds of the Formula I and III-IX, the invention also includes solvates, pharmaceutically acceptable prodrugs, pharmaceutically active metabolites, and pharmaceutically acceptable salts of such compounds.

The term “solvate” refers to an aggregate of a molecule with one or more solvent molecules.

A “pharmaceutically acceptable prodrug” is a compound that may be converted under physiological conditions or by solvolysis to the specified compound or to a pharmaceutically acceptable salt of such compound.

A “pharmaceutically active metabolite” is a pharmacologically active product produced through metabolism in the body of a specified compound or salt thereof. Metabolites of a compound may be identified using routine techniques known in the art and their activities determined using tests such as those described herein.

Prodrugs and active metabolites of a compound may be identified using routine techniques known in the art. Various forms of prodrugs are known in the art. For examples of such prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs”, by H. Bundgaard p. 113-191 (1991); c) H. Bundgaard, Advanced Drug Delivery Reviews, 8, 1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

A “pharmaceutically acceptable salt” is a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable sale. Examples of pharmaceutically acceptable salts include those salts prepared by reaction of the compounds of the present invention with a mineral or organic acid or an inorganic base, such salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitromenzoates, hydroxybenzoates, methoxybenzoates, phthalates, sulfonates, xylenesulfonates, pheylacetates, phenylpropionates, phenylbutyrates, citrates, lactates, γ-hydroxybutyrates, glycollates, tartrates, methanesulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alphahydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

According to the present invention, suitable methods of administering the therapeutic composition of the present invention to a patient include any route of in vivo administration that is suitable for delivering the composition into a patient. The preferred routes of administration will be apparent to those of skill in the art, depending on the type of condition to be prevented or treated, and/or the target cell population. Preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue.

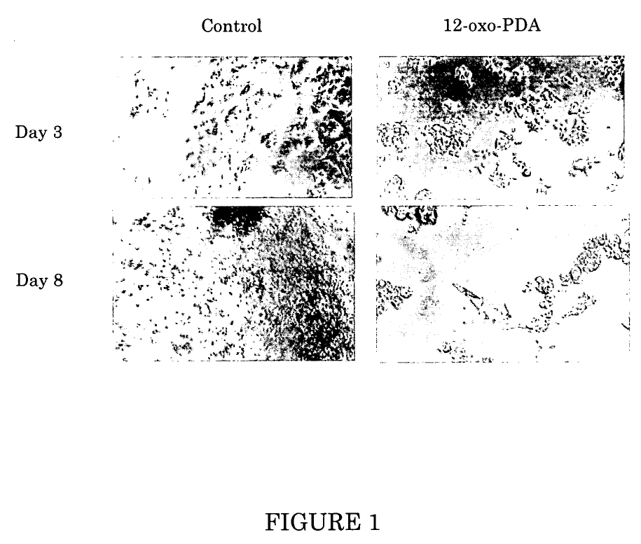

As described in further detail below and illustrated in FIGS. 1-14, the present invention demonstrates that 12-oxo-PDA (I) arrests cell growth and induces cell death of actively and exponentially proliferating high-grade metastatic human breast cancer cells (T-47D), lung cancer cells (CRL-5985) and malignant human B-cell lymphoma cells (CRL-2632) in a dose dependant manner. Further, the invention demonstrates that at similar concentrations, jasmonic acid fails to be as effective.

It was also observed that exposure of the cancer cells to 12-oxo-PDA resulted in significant morphological changes whereby the cancer cells ceased three-dimensional growth patterns and became more diffuse and single layered. Additionally, 12-oxo-PDA resulted in lipid accumulation in the cytoplasm of the cancer cells as shown by Oil Red 0 staining. Importantly, the data suggests that 12-oxo-PDA (and the related compounds) is not toxic to the cancer cells. Rather, it appears that 12-oxo-PDA (and the related compounds) irreversibly interferes with the cellular mechanisms involved in cell growth, differentiation and the interaction between the cells with the extracellular matrix. Indeed, removal of 12-oxo-PDA from the culture mediums did not restore cell growth.

Treatment with 12-oxo-PDA also resulted in inhibition of cyclin D1 expression and inhibition of the hyperphosphorylation of retinoblastoma tumor suppressor protein (“Rb”) in T-47D metastatic human breast cancer cells.

Accordingly, one aspect of the invention is the use of 12-oxo-PDA and the related compounds disclosed herein as part of a method for treating cancer and other conditions characterized by uncontrolled or otherwise excessive cell proliferation.

The invention is further illustrated by the following non-limited examples. All scientific and technical terms have the meanings as understood by one with ordinary skill in the art. The specific examples which follow illustrate the methods in which the compositions of the present invention may be used and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLES

In the following examples, all referenced cell lines were purchased from ATCC. T47D cells were maintained in Dulbecco's Modified Eagle medium (DMEM) (purchased from Life Technologies, Inc.) supplemented with 10% fetal calf serum (purchased from Life Technologies, Inc.), insulin (5 μg/ml), and antibiotics. CRL-5985, CRL-2632 and Raji cells were maintained in RPMI-1640 medium supplemented with 10% fetal calf serum (purchased from Life Technologies, Inc.) and antibiotics. Western blot analysis was performed by using phospho-serine 807/811-Rb, Rb, cyclin D1, cyclin D3, cdk-2, cdk-4, bcl-2, phospho-p38, bax, bak, PARP (cell signaling technology), MCL-1 (purchased from Biosource International) and beta-tubulin antibodies (purchased from Becton-Dickenson). Proteins were separated using an 8-10% SDS-polyacrylamide gel electrophoresis (SDS-PAGE), transferred to Immobilon-P membranes and immunoblotted with antibodies using the ECL detection system.

For real-time RT-PCR experiments T-47D cells were treated with ethanol (control) or with 25 uM 12-OPDA for 48 hours. Total RNA was isolated using TRIzol reagent (Invitrogen, Carlsbad, Calif.) followed by purification on RNeasy columns (Qiagen, Palo Alto, Calif.). For quantitative real-time RT-PCR analyses, total RNA from each sample was reverse transcribed with MuLV reverse transcriptase (Applied Biosystems, Foster City, Calif.) and random hexamer primers. The SYBR Green PCR Kit (Applied Biosystems) was used for quantitative real-time RT-PCR analysis. The primers for human ubuquitin, cyclin D1, p27 and c-fos were designed using Primer Express software (Applied Biosystems). 12-oxo-PDA was purchased from Larodon Fine Chemicals AB, Sweden. Jasmonic acid was purchased from Sigma-Aldrich Co. The indanoyl amino acid conjugate of coronatine (i.e., synthetic 6-azido-1-oxo-indanoyl isoleucine) was obtained from Dr. Wilhelm Boland (Max Planck Institute, Germany).

### Example 1

### Method for treating breast cancer cells with 12-oxo-PDA (I)

T-47D human breast ductal adenocarcinoma cells were treated with 12-oxo-PDA and measured at three and eight-day intervals. As can be seen in FIG. 1, there is a significant decrease in cell growth and accumulation in the cells treated with 12-oxo-PDA compared to the untreated control sample.

### Example 2

### Method for Treating Lung Cancer Cells with 12-oxo-PDA (I)

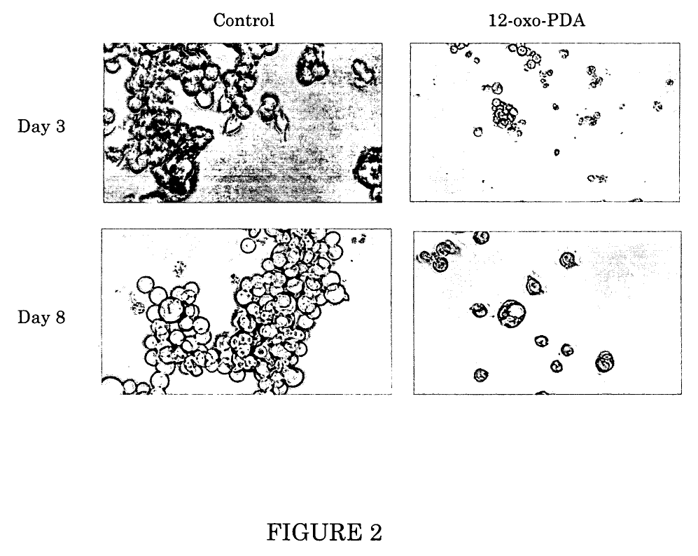

CRL-5985 human lung adenocarcinoma cells were treated with 12-oxo-PDA and measured at three and eight-day intervals. FIG. 2, which shows a 200× magnification of the treated cells, demonstrates that in comparison to a control sample there is a significant decrease in cell growth and morphological changes in the cells treated with 12-oxo-PDA, whereby the cancer cells ceased three-dimensional growth patterns and became more diffuse and single layered.

### Example 3

### Method of Treating CRL-5985 Human Lung Adenocarcinoma with 12-oxo-PDA (I)

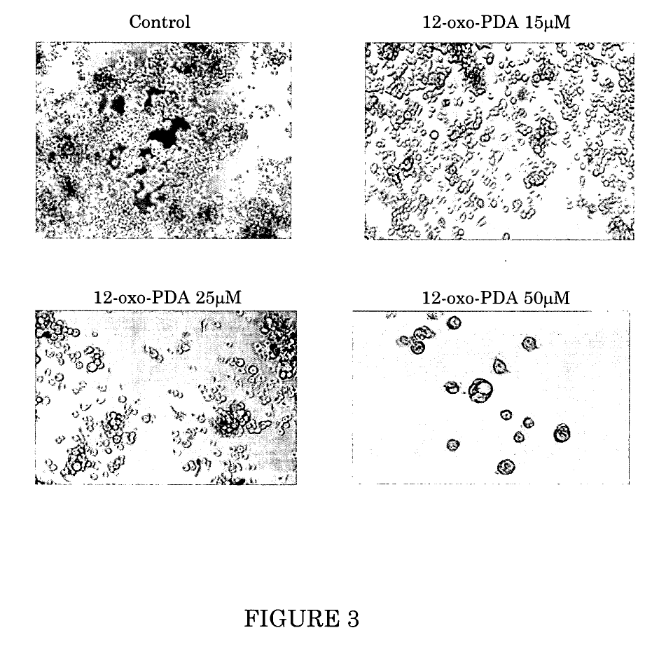

Lung cancer cells were treated with concentrations of 12-oxo-PDA ranging from 15 μM to 50 μM (versus an untreated control sample). After eight days of treatment at the various concentrations, a 200× magnification of the cells (FIG. 3) revealed a decrease in cell growth proportional to the concentration of 12-oxo-PDA supplied to the cells (i.e., treatment with higher concentrations of 12-oxo-PDA resulted in a greater decrease in cell growth).

### Example 4

### Method for Treating CRL-2632 Human Malignant Diffuse Large B-cell Lymphoma Cells with 12-oxo-PDA (I)

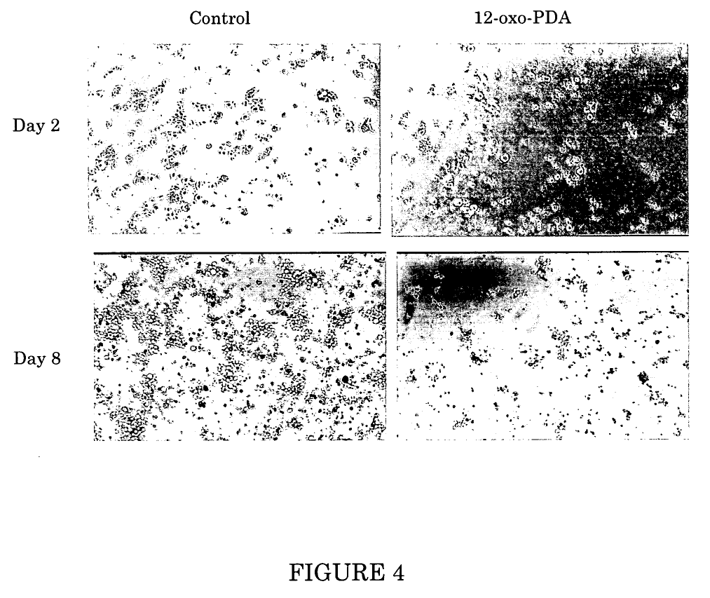

FIG. 4 is a 200× magnification of human malignant diffuse large B-cell lymphoma cells showing morphological changes at two and eight-day intervals resulting from treatment with 12-oxo-PDA versus a control sample.

### Example 5

### Effect of 12-oxo-PDA (I) on Cell Viability and Cell Growth

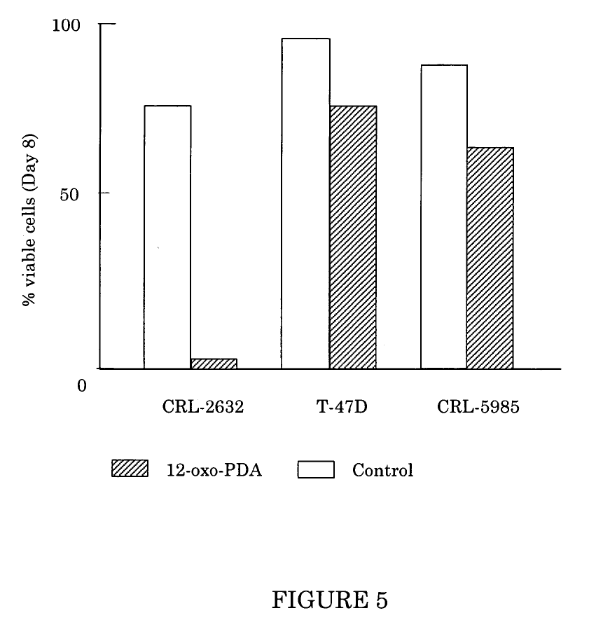

Cell lines CRL-2632 (B-cell lymphoma cells), CRL-5985 (human lung adenocarcinoma cells) and T-47D (human breast ductal adenocarcinoma cells) were exposed to 12-OPDA over an eight-day period. Cell viability and cell growth were measured versus controls. As shown in FIG. 5, 12-oxo-PDA had a significant inhibitory effect on cell viability, as determined by trypan blue exclusion assay. FIG. 5 illustrates that 12-oxo-PDA was particularly effective at affecting the viability of the treated CRL-2632 cells.

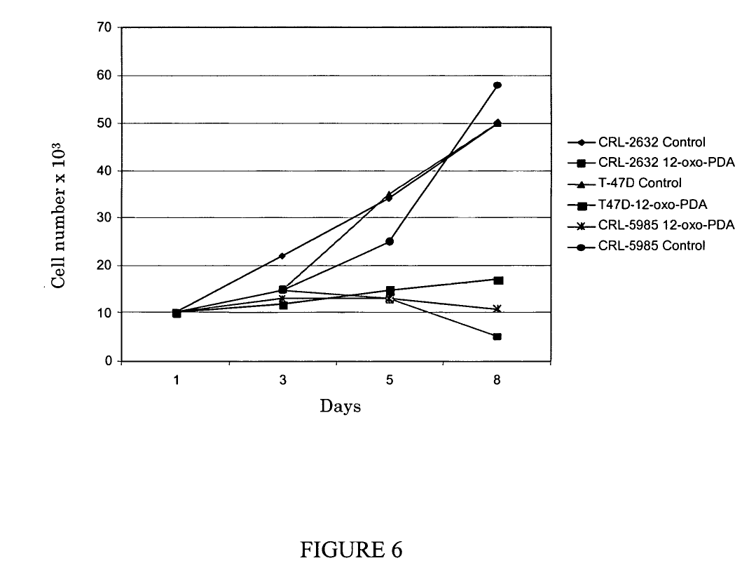

As demonstrated in FIG. 6, it was observed that the CRL-2632 cells had approximately a ten-fold decrease in total cell number following treatment with 12-oxo-PDA (versus untreated samples over the same period of time). Similarly, the T-47D cells exhibited an approximate three-fold decrease in cell number while the CRL-5985 cells demonstrated a five-fold decrease after treatment with 12-oxo-PDA (versus untreated samples over the same period of time).

### Example 6

### 12-Oxo-PDA (I) Versus Jasmonic Acid (II) on Cell Growth of Breast Cancer Cells

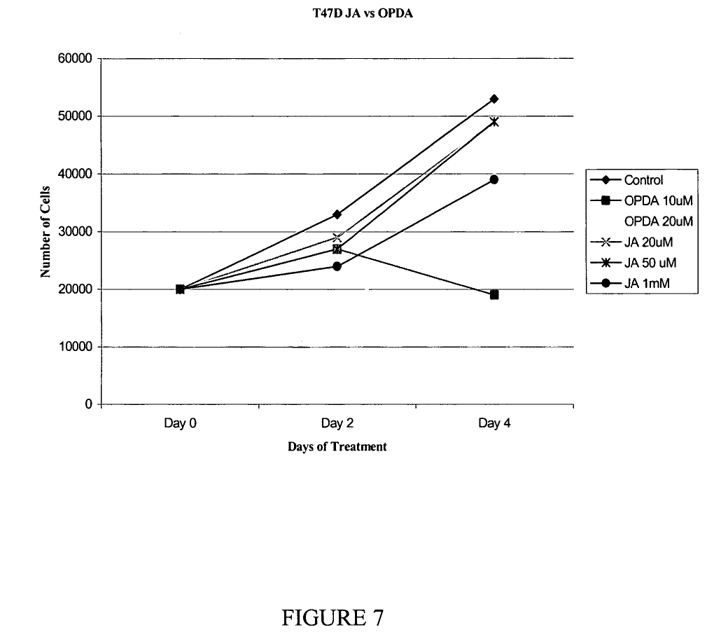

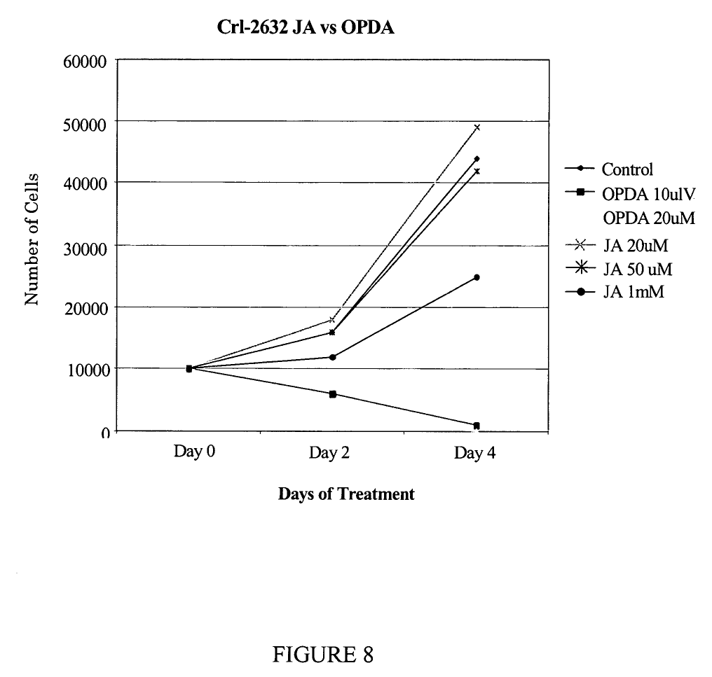

The effects on cell growth and number for T-47D human breast cancer cells and CRL-2632 human malignant diffuse large B-cell lymphoma cells following treatment with 12-oxo-PDA and jasmonic acid (“JA”) over a four-day period were measured. As demonstrated in FIGS. 7 and 8, there was a considerable decrease in total cell number among T-47D and CRL-2632 cells, respectively, treated with 12-oxo-PDA compared to those treated with JA. In fact, treatment with 12-oxo-PDA resulted in a decrease in total cell count. Conversely, cells treated with JA, although showing some growth inhibition relative to an untreated control, nonetheless had a net increase in total tumor cell number during the treatment period.

### Example 7

### Cyclin D1 Expression is Inhibited by 12-oxo-PDA (I)

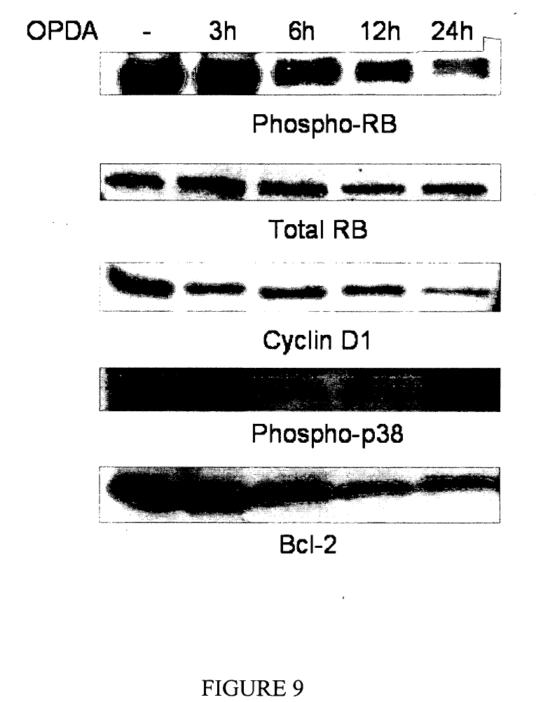

Treatment of T-47D metastatic human breast cancer cells with 12-oxo-PDA (20 μM) results in inhibition of cyclin D1 expression and of the hyperphosphorylation of retinoblastoma tumor suppressor protein (“Rb”). Hyperphosphorylation of Rb is a critical process and component for regulating the cell cycle. The phosphorylation of Rb enables cells to pass through particular cell cycle checkpoints and to enter the S phase of cell cycle. FIG. 9 demonstrates that treatment of T-47D cells with 12-oxo-PDA dramatically inhibited Rb phosphorylation at the 807 and 811 serine residues (it is known that the 807 and 811 serine residues of Rb are principally phosphorylated by the cyclin D1-CDK4 complex) as determined on Western blot analysis using a phospho-specific Rb antibody. FIG. 9 further demonstrates that the inhibition of Rb phosphorylation correlates with decreased cyclin D1 expression. Despite these inhibitory effects, total Rb remained unchanged. Similarly, expression of β-tubulin and phosphorylation of p38 MAPK were unaffected.

### Example 8

### Measurement of Regulatory/Inhibitory Effects of 12-oxo-PDA (I)

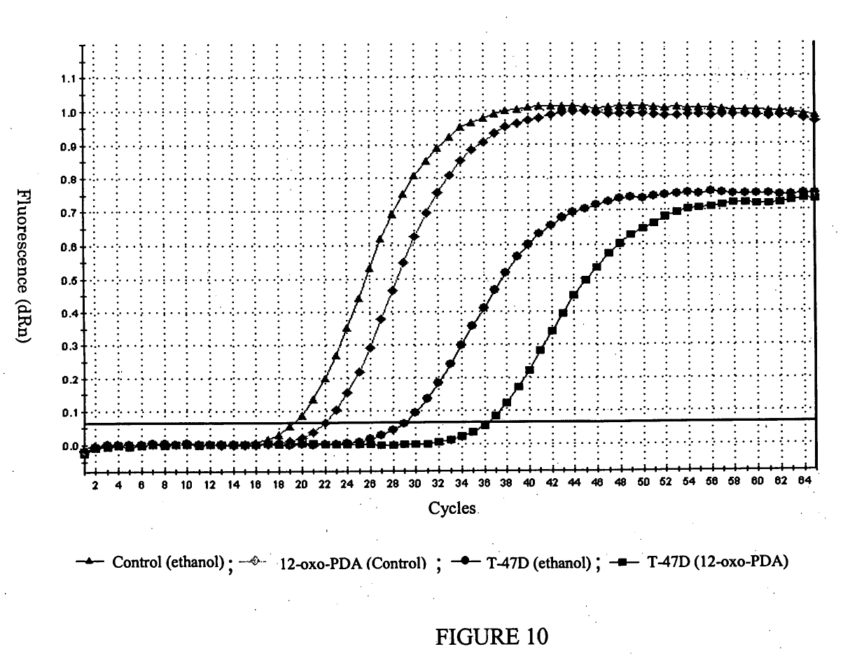

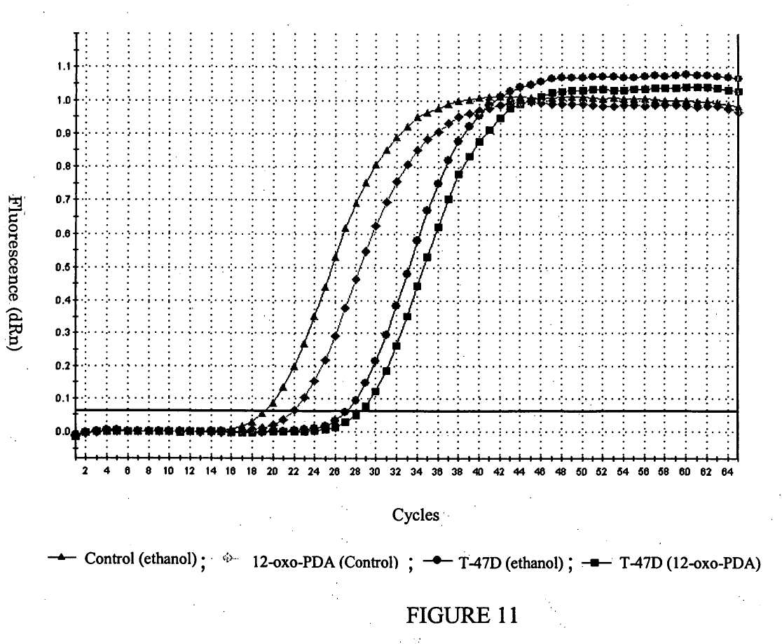

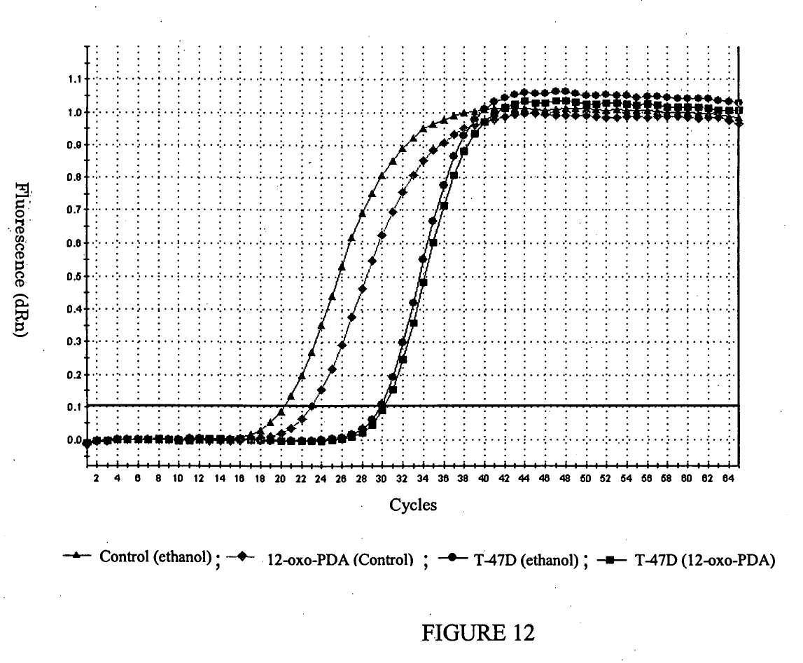

T-47D cells were exposed to 12-oxo-PDA in an effort to determine the effect of 12-oxo-PDA on gene expression. FIGS. 10, 11 and 12 show 12-OPDA mediated regulation of cyclin D1, p27 and c-fos mRNA levels, respectively, in T-47D cells compared to ethanol treatment alone. As demonstrated in FIG. 10, treatment with 12-oxo-PDA specifically down regulates the expression of cyclin D1 mRNA, but has no significant effect on expression of p27 (FIG. 11) and C-FOS m RNA (FIG. 12) in the T-47D cells. In fact, a greater than twenty-fold decrease in cyclin D1 mRNA levels was observed in cells treated with 12-oxo-PDA relative to untreated T-47D cells. Conversely, no significant changes were observed in the p27 and C-FOS mRNA levels upon 12-oxo-PDA treatment. Thus, it appears that 12-oxo-PDA has a direct regulatory/inhibitory effect on the cyclin D1 and Rb protein. Ubiquitine expression in ethanol and 12-OPDA treated cells has been used as internal control in each amplification experiment (left curves).

### Example 9

### Apoptosis Induced by 12-oxo-PDA (I)

Regulated cell apoptosis is essential for the development and maintenance of the immune system. The highly regulated anti-apoptotic MCL-1 protein appears to enhance short-term survival and functions in genotoxic cell death. Removal of MCL-1 has been shown to cause a profound reduction in B and T lymphocyte cells in mice. Thus, deletion of MCL-1 during early lymphocyte differentiation increases apoptosis and arrests the development of B and T-lymphocytes. In this regard, one hallmark of apoptosis is activation of the caspases (e.g., caspase-3, caspase-6, and caspase-7). Activation of caspases leads to the cleavage of a 11 8-kDa PARP protein into an 89-kDa fragment.

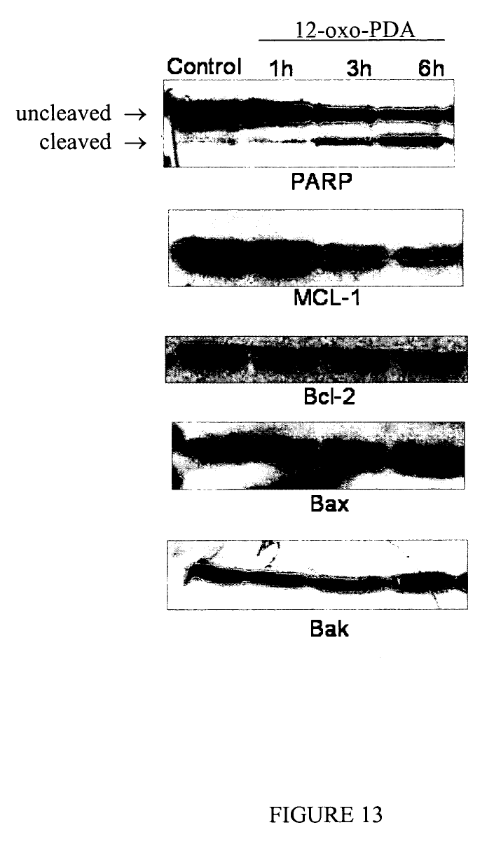

In FIG. 13, CRL-2632 diffuse large B-cell lymphoma cells were treated with 12-oxo-PDA to demonstrate that 12-oxo-PDA induces apoptosis and inhibits MCL-1 expression. Cells were incubated in the presence of 12-oxo-PDA (20 μM) for between one and six hours. The cells were then washed and lysed to extract total proteins. Thereafter, 30 μg extracts were resolved on 8% SDS-PAGE gel and probed with anti-PARP, anti-MCL-1, anti-bcl-2, anti-bax and anti-bak antibodies. As shown in FIG. 12, immunoblot analysis of the extracts from cells treated with 12-oxo-PDA showed a time-dependent cleavage of PARP (whereas untreated cells showed no PARP cleavage). Similarly, FIG. 13 illustrates the effects of 12-oxo-PDA treatment on apoptosis related proteins (i.e., Bcl-2, MCL-1, Bax and Bak proteins). Specifically, 12-oxo-PDA inhibited the expression of MCL-1, but had no effect on the expression of Bcl-2, Bax and/or Bak proteins in the CRL-2632 cells.

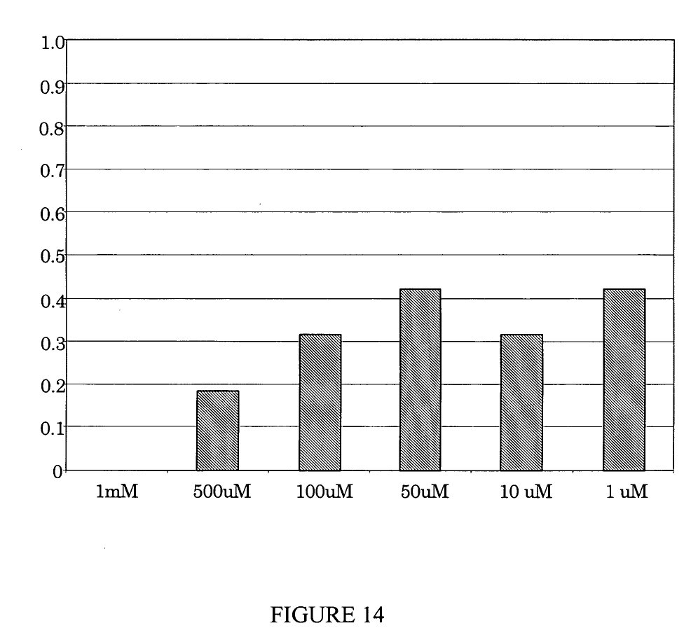

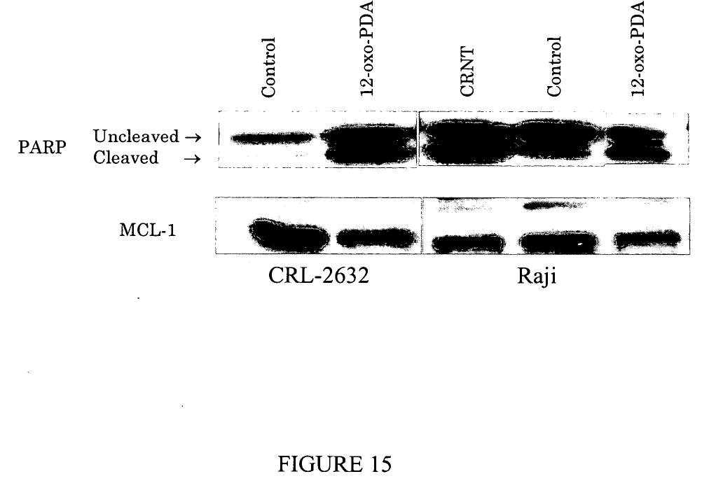

Synthetic coronatine (“CRNT”) can inhibit growth of CRL-2632 cells (FIG. 14) and also down regulate the expression of the MCL-1 protein and induce apoptosis (FIG. 15). CRL-2632 diffuse large B-cell lymphoma cells (and Burkitt's lymphoma cells (“Raji”)) were treated with 12-oxo-PDA and a synthetic coronatine analog (0.5 mM). Both 12-oxo-PDA and synthetic coronatine arrest growth and induce apoptosis in the CRL-2632 and Raji cells, as demonstrated in FIG. 15. Furthermore, both 12-oxo-PDA and the synthetic coronatine down-regulated expression of MCL-1 in the treated lymphoma cells. As such, the foregoing data further suggests that both 12-oxo-PDA and coronatine induce apoptosis in high-grade lymphoma cells and inhibit MCL-1 protein expression by activation of caspase pathways.

### Example 10

### Induction of Tubulin Polymerization

Mitotic spindles are critical elements in a variety of fundamental cellular functions including, for example, chemotaxis, membrane and intracellular scaffolding, transport, secretory processes, regulatory of cellular motility and cell division. Microtubules play essential roles in the formation, operation and regulation of the mitotic spindles. As such, disruption of the regulatory functioning and apparatus of the microtubules can induce cell-cycle arrest during the M phase of cellular development and trigger signals to induce programmed cell death. In this regard, microtubule inhibitors interfere with the tubulin dynamics of tubulin polymerization and depolymerization and result in the inhibition of cell division. For example, anti-mitotic drugs (e.g., taxanes and Vinca alkaloids) have been used to treat various kinds of human cancers. The taxanes, including paclitaxel and docetaxel, stabilize microtubules and induce net microtubule polymerization and are effective in the treatment of breast, lung, ovarian, bladder, head and neck cancers. The Vinca alkaloids, such as vincristine, vinblastine and vinorelbine, prevent normal polymerization of microtubules and are important in treating leukemia, lymphoma, small cell lung cancer and other malignancies.

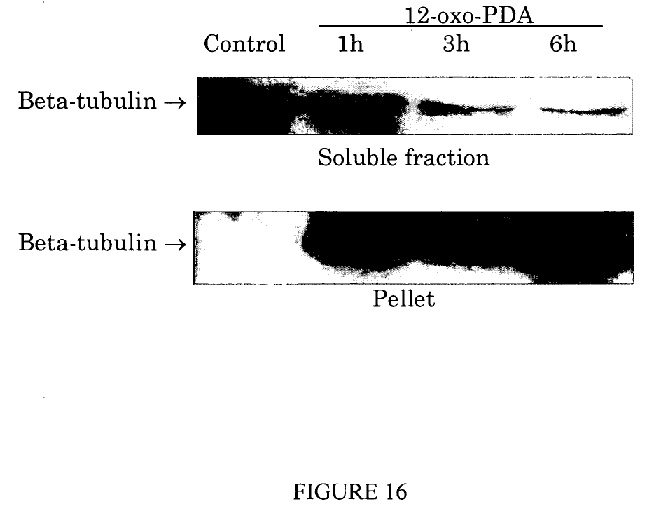

In FIG. 16 it is demonstrated that 12-oxo-PDA induces tubulin polymerization in CRL-2632 cells. In particular, it was observed that treatment of exponentially growing CRL-2632 cells with 12-oxo-PDA (20 μM) for between one and six hours resulted in the loss of tubulin in soluble but accumulation in insoluble fractions (free tubulin and microtubule fractions (pellet) were isolated and analyzed by Western blot). The increase in insoluble tubulin fractions indicates polymerization of tubulin in treated cells. From this data it appears that 12-oxo-PDA may interfere with tubulin polymerization and depolymerization processes and thus inhibit cancer cell division.

In view of the foregoing results, it should be noted that the above-treated cells each attribute their malignant behavior to different transformation processes and mechanisms. These varying processes include aberrant ras oncogene activity in CRL-5985 human lung adenocarcinoma cells, a non-functional p53 pathway in T-47D breast ductal carcinoma cells and the excessive expression of bcl-2 anti-apoptotic factor in CRL-2632 human diffuse large B-cell lymphoma cells. Despite these various mechanisms, 12-oxo-PDA and its related compounds nonetheless have an inhibitory effect on cell growth. Thus, it is possible that the inhibitory effects of 12-oxo-PDA and its related compounds may be independent of the particular mechanism involved in the malignant transformation of cells. As such, 12-oxo-PDA and its related compounds may be particularly useful for treating malignant cells that have shown resistance to other known chemotherapeutic agents (and especially resistances to cell cycle and apoptosis regulatory protein agents such as those affecting p53 and/or bcl-2).

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A method of controlling proliferative cells in a subject, comprising administering a therapeutically effective amount of at least one compound having the formula: 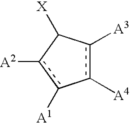
wherein
—is an optional double bond;
A¹ and A² are independently H, Zₘ-OR⁶, oxo, halo, Zₘ-CN, Zₘ-NO₂, azido, Zₘ-NR⁶R⁷, Zₘ-COOR⁶, Zₘ-CONR⁶R⁷, Zₘ-C(═O)R⁶, Zₘ-OC(═O)R⁶, alkyl, allyl, alkenyl, alkynyl, heteroalkyl, heteroallyl, heteroalkenyl, heteroalkynyl, heteroalkoxy, thiol, thioalkyl, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted;
A³ and A⁴ are independently alkyl, allyl, alkenyl, alkynyl, heteroalkyl, heteroallyl, heteroalkenyl, heteroalkynyl, alkoxy, heteroalkoxy, Zₘ-cycloalkyl wherein said cycloalkyl is saturated or partially unsaturated, Zₘ-heterocycloalkyl wherein said heterocycloalkyl is saturated or partially unsaturated, Zₘ-Ar, Zₘ-O—R⁶, Zₘ-SR⁶, Zₘ-NR⁶R⁷, Zₘ-C(═O)R⁶, Zₘ-OC(═O)R⁶, Zₘ-C(═O)OR⁶, Zₘ-(C═O)NR⁶R⁷, or Zₘ-NHC(═O)R⁶, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted and wherein at least one of A³ or A⁴ is at least three atoms in length;
or A³ and A⁴ together with the atoms to which they are both attached form a substituted or unsubstituted saturated or partially unsaturated ring or a substituted or unsubstituted aromatic ring having at least five atoms, wherein one or more of the atoms is optionally a heteroatom;
R⁶ and R⁷ are independently H, Zₘ-OR⁶, alkyl, allyl, alkenyl, alkynyl, heteroalkyl, heteroallyl, heteroalkenyl, heteroalkynyl, heteroalkoxy, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted;
X is OR⁶, oxo, heteroalkoxy, O-glucosyl, thiol, thioalkyl, NR⁶R⁷, halo, CN, NO₂, or azido;
Ar is aryl or heteroaryl;
Z is CH₂; and
m is an integer between 0 and 10.

2. The method of claim 1, wherein A³ and A⁴ are independently 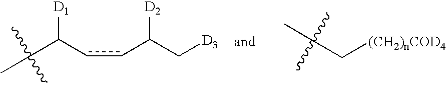
wherein
n is 3, 4, 5, 6, 7, 8, 9, or 10;
D₁, D₂ and D₃ are independently H, Zₘ-OR⁶, Zₘ-O-glucosyl, heteroalkoxy, thiol, thioalkyl, Zₘ-NR⁶R⁷, halo, Zₘ-CN, Zₘ-NO₂, or azido;
D₄ is H, Zₘ-OR⁶, O-glucosyl, imino, halo, Zₘ-CN, Zₘ-NO₂, azido, Zₘ-C(═O)H, Zₘ-NR⁶R⁷, Zₘ-COOR⁶, ZₘCONR⁶R⁷, Zₘ-C(═O)R⁶, Zₘ-OC(═O)R⁶, alkyl, allyl, alkenyl, alkynyl, heteroalkyl, heteroallyl, heteroalkenyl, heteroalkynyl, heteroalkoxy, thiol, thioalkyl, Zₘ-cycloalkyl wherein said cycloalkyl is saturated or partially unsaturated, Zₘ-heterocycloalkyl wherein said heterocycloalkyl is saturated or partially unsaturated, or Zₘ-Ar¹, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar¹ may be substituted or unsubstituted;
or D₄ and X, or D₄ and D₃ together form a lactone; and
m is an integer between 0 and 10.

3. The method of claim 1, wherein A³ and A⁴ are independently

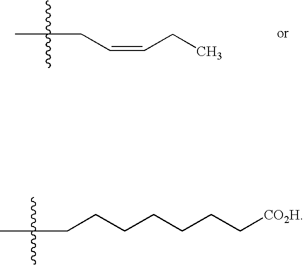

4. The method of claim 1, wherein the compound is

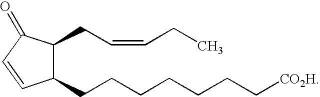

5. The method of claim 1, wherein A³ and A⁴ together form a six-member ring.

6. The method of claim 5, wherein said six-member ring contains at least one carbon-carbon multiple bond.

7. The method of claim 5, wherein said six-member ring is aromatic.

8. The method of claim 5, wherein said six-member ring contains at least one additional substituent group.

9. The method of claim 8, wherein said at least one additional substituent group is selected from the group of H, OR⁶, oxo, halo, CN, NO₂, azido, NR⁶R⁷, COOR⁶, CONR⁶R⁷, C(═O)R⁶, OC(═O)R⁶, alkyl, allyl, alkenyl, alkynyl, heteroalkyl, heteroallyl, heteroalkenyl, heteroalkynyl, heteroalkoxy, thiol, thioalkyl, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar may be substituted or unsubstituted.

10. The method of claim 1, wherein the compound is 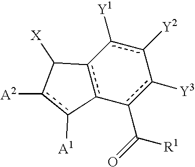
wherein R¹ is 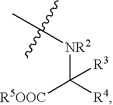
R², R³, R⁴ and R⁵ are independently H, Zₘ-OR⁶, alkyl, allyl, alkenyl, alkynyl, heteroalkyl, heteroallyl, heteroalkenyl, heteroalkynyl, heteroalkoxy, Zₘ-NR⁶R⁷, Zₘ-COOR⁶, Zₘ-CONR⁶R⁷, Zₘ-C(═O)R⁶, Zₘ-OC(═O)R⁶, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₙ-heterocycloalkyl, and Zₘ-Ar may be substituted or unsubstituted,
or R³ and R⁴ together with the atoms to which they are both attached form a saturated or partially unsaturated ring, wherein said saturated ring or partially unsaturated ring may be substituted or unsubstituted; and
Y¹, Y², and Y³ are independently H, Zₘ-OR⁶, alkyl, allyl, alkenyl, alkynyl, heteroalkyl, heteroallyl, heteroalkenyl, heteroalkynyl, heteroalkoxy, Zₘ-NR⁶R⁷, Zₘ-COOR⁶, Zₘ-CONR⁶R⁷, Zₘ-C(═O)R⁶, Zₘ-OC(═O)R⁶, Zₘ-cycloalkyl wherein said cycloalkyl is saturated or partially unsaturated, Zₘ-heterocycloalkyl wherein said heterocycloalkyl is saturated or partially unsaturated, or Zₘ-Ar, wherein said alkyl, allyl, alkenyl, alkynyl, heteroalkyl, heteroallyl, heteroalkenyl, heteroalkynyl, heteroalkoxy, Zₘ-cycloalkyl, Zₙ-heterocycloalkyl, and Zₘ-Ar may be substituted or unsubstituted.

11. The method of claim 10, wherein R¹ is a substituted or unsubstituted natural or unnatural amino acid.

12. The method of claim 11, wherein R¹ is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

13. The method of claim 11, wherein R¹ is 4-hydroxyproline, hydroxylysine, demosine, isodemosine, 3-methylhistidine, norvaline, beta-alanine, gamma-aminobutyric acid, cirtulline, homocysteine, homoserine, omithine and methionine sulfone.

14. The method of claim 10, wherein the compound is

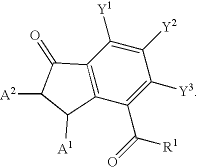

15. The method of claim 14, wherein said compound is

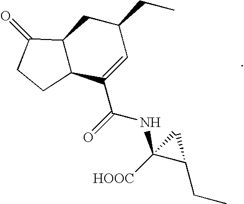

16. The method of claim 10, wherein said compound is

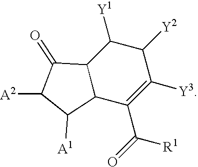

17. The method of claim 16, wherein said compound is

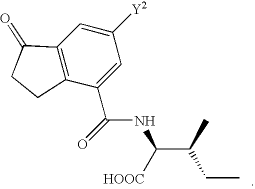

18. The method of claim 1, wherein said subject has cancer.

19. The method of claim 1, wherein said cancer is ovarian cancer.

20. The method of claim 1, wherein said cancer is breast cancer.

21. The method of claim 1, wherein said cancer is lung cancer.

22. The method of claim 1, wherein said cancer is lymphoma.

23. The method of claim 1, wherein said method of treatment further comprises at least one of an hourly administration, a daily administration, a weekly administration, or a monthly administration of said at least one composition.

24. The method of claim 1, wherein said administration comprises oral administration of said at least one composition.

25. The method of claim 1, wherein said administration comprises injection of said at least one composition.

26. The method of claim 1, wherein said administration comprises intravenous administration of said at least one composition.

27. The method of claim 1, wherein said subject is an animal.

28. The method of claim 1, wherein said subject is a human.

29. A method for controlling proliferative cells in a subject, comprising supplying to said subject at least one compound of the formula:

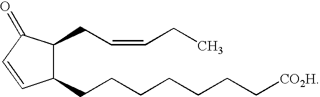

30. A method for controlling proliferative cells in a subject, comprising supplying to said subject a compound of the formula:

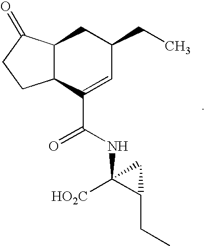

31. A method for controlling proliferative cells in a subject, comprising supplying to said subject a compound of the formula:

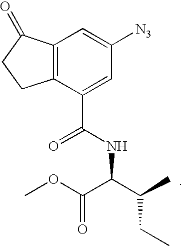

32. A method for conducting a clinical trial comprising supplying to a subject at least one compound of the formula: 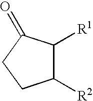
wherein said composition contains at least one additional carbon-carbon multiple bond; and
wherein one or both of R¹ and R² define a structure selected from the group consisting of (a) at least one substituent selected from the group of hydrogen, alkyl, allyl, alkenyl, alkynyl, heteroalkyl, heteroallyl, heteroalkenyl, heteroalkynyl, alkoxy, heteroalkoxy and (b) a second ring structure of at least five atoms.

33. The method of claim 1, wherein A⁴ is 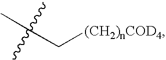
n is 3,4, 5, 6, 7, 8, 9, or 10; and
D₄ is H, Zₘ-OR⁶, O-glucosyl, imino, halo, Zₘ-CN, Zₘ-NO₂, azido, Zₘ-C(═O)H, Zₘ-NR⁶R⁷, Zₘ-COOR⁶, Zₘ-CONR⁶R⁷, Zₘ-C(═O)R⁶, Zₘ-OC(═O)R⁶, alkyl, allyl, alkenyl, alkynyl, heteroalkyl, heteroallyl, heteroalkenyl, heteroalkynyl, heteroalkoxy, thiol, thioalkyl, Zₘ-cycloalkyl wherein said cycloalkyl is saturated or partially unsaturated, Zₘ-heterocycloalkyl wherein said heterocycloalkyl is saturated or partially unsaturated, or Zₘ-Ar¹, wherein said alkyl, allyl, alkenyl, alkynyl, heteroalkyl, heteroallyl, heteroalkenyl, heteroalkynyl, heteroalkoxy, Zₘ-cycloalkyl, Zₘ-heterocycloalkyl, and Zₘ-Ar¹ may be substituted or unsubstituted.

34. A method of controlling proliferative cells in a subject, comprising administering a therapeutically effective amount of at least one compound having the formula:

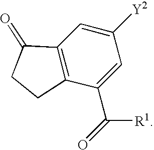

35. The method of claim 34, wherein R¹ is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

36. The method of claim 34, wherein R¹ is 4-hydroxyproline, hydroxylysine, demosine, isodemosine, 3-methylhistidine, norvaline, beta-alanine, gamma-aminobutyric acid, cirtulline, homocysteine, homoserine, omithine and methionine sulfone.

37. A pharmaceutical composition for controlling proliferative cells in a subject, comprising a therapeutically effective amount of a compound having the formula: 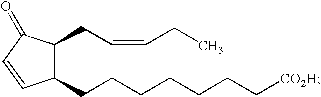
and a pharmaceutically acceptable carrier.

38. A pharmaceutical composition for controlling proliferative cells in a subject, comprising a therapeutically effective amount of a compound having the formula: 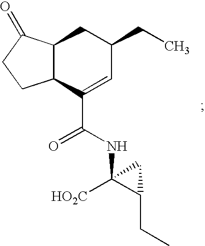
and a pharmaceutically acceptable carrier.

39. A pharmaceutical composition for controlling proliferative cells in a subject, comprising a therapeutically effective amount of a compound having the formula: 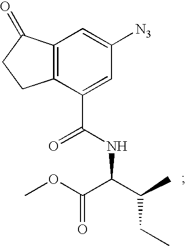
and a pharmaceutically acceptable carrier.
